(19) United States
(12) Patent Application Publication
(10) Pub. No.: US 2022/0273105 A1
(43) Pub. Date: Sep. 1, 2022

(54) HEAT AND MASSAGE SYSTEM FOR A FURNITURE PIECE AND A METHOD OF OPERATING THE SAME

(71) Applicant: HHC CHANGZHOU CORPORATION, CHANGZHOU (CN)

(72) Inventors: CHIH HSIUNG LIU, BENONI (ZA); MEI JUN XU, JIUJIANG (CN); XIAO PING RAN, XICHONG (CN)

(21) Appl. No.: 17/187,967

(22) Filed: Mar. 1, 2021

(51) Int. Cl. A47C 7/74; A47C 7/46; A47C 7/38; A47C 7/72; A61H 9/00; A61H 7/00

(52) U.S. Cl. CPC A47C 7/748; A47C 7/46; A47C 7/38; A47C 7/725; A61H 9/0078; A61H 7/004; A61H 2201/0207; A61H 2201/0149; A61H 2201/5025; A61H 2205/081; A61H 2201/5043; A61H 2201/1238; A61H 2201/164; A61H 2201/1604; A61H 2201/5015; A61H 2201/0188

(57) ABSTRACT

The present invention relates to a piece of furniture within which a massage system has been incorporated so as to provide a person, sifting in the piece of furniture, with massage capabilities so as to relieve pressure, stress, and/or tension. The system comprises a lumbar actuator which is adapted to engage a lumbar portion of the piece of furniture, wherein the lumbar actuator will be cyclically activated in accordance with single or multiple frequencies or modes of operation so as to provide the person, sifting in the piece of furniture, with varying degrees of massage as may be desired by the user or person sitting in the piece of furniture. In addition, inflatable/deflatable air bladders are located at strategically placed regions upon the back and lumbar portions of the piece of furniture so as to provide additional massage therapy, and still further, heating elements are located within the back, lumbar, and seat portions of the piece of furniture so as to provide heat therapy to a person's back, lumbar regions, and hip regions of the person's body.

### FIELD OF THE INVENTION

The present invention relates generally to furniture, and more particularly to a household or office furniture piece which may comprise, for example, a lounger chair, an armchair, a sofa, or similar type piece of furniture, within which a massage system has been incorporated so as to provide a person, sitting in the chair, sofa, or similar type piece of furniture, with massage capabilities so as to relieve pressure, stress, and/or tension.

### BACKGROUND OF THE INVENTION

In the current global economy, competition is fierce, people are busy working, and there are always a multitude of production and shipping deadlines to be met in accordance with various different product purchasing contracts. In short, working people and business executives at all levels of business are under a great deal of pressure, stress, and tension. In order to effectively relieve such pressure, stress, and tension, which can, in turn, result in fatigue which, of course, is counter-productive in achieving work-related goals, many people are turning to massage therapy. Conventional chairs and other furniture pieces, designed to provide some type of massage, usually have mechanisms incorporated therein which provide variations of kneading, vibration, knocking, and the like, and have not really proven to be reliable in delivering the type of massage that is desired in order to reduce pressure, stress, tension, and the like. The reason for this is that such conventional chairs and other furniture pieces do not actually target the correct part of the body and provide the necessary massage to that region of the body. One of the most important parts of the body to be massaged in order to effectively reduce pressure, stress, tension, and the like, is a person's back, and more particularly, the lumbar region of the back. In addition, other strategically located regions of the body are particularly susceptible to heat and massage for therapeutic purposes, using, for example, heating pads and air bladder mechanisms in addition to the previously noted cyclic lumbar massage mechanism, such that enhanced massage therapy may in fact be achieved.

A need therefore exists in the art for a new and improved piece of furniture. An additional need exists in the art for a new and improved piece of furniture that can provide several different types of massage that can in fact help to reduce pressure, stress, tension, and the like. A further need exists in the art for a new and improved piece of furniture that can provide several different types of massage that can in fact help to reduce pressure, stress, tension, and the like as a result of the different massage mechanisms incorporated within the piece of furniture that target different regions of the body. A still further need exists in the art for a new and improved piece of furniture that can provide several different types of massage that can in fact help to reduce pressure, stress, tension, and the like as a result of the different massage mechanisms incorporated within the piece of furniture that target different regions of the body such as, for example, the lumbar region of a person's back, as well as lower back, mid-back, and upper back regions of the person's back.

### OVERALL OBJECTIVES OF THE PRESENT INVENTION

An overall objective of the present invention is to provide a new and improved piece of furniture. An additional overall objective of the present invention is to provide a new and improved piece of furniture that can provide several different types of massage that can in fact help to reduce pressure, stress, tension, and the like. A further overall objective of the present invention is to provide a new and improved piece of furniture that can provide several different types of massage that can in fact help to reduce pressure, stress, tension, and the like as a result of the different massage mechanisms incorporated within the piece of furniture that target different regions of the body. A still further overall objective of the present invention is to provide a new and improved piece of furniture that can provide several different types of massage that can in fact help to reduce pressure, stress, tension, and the like as a result of the different massage mechanisms incorporated within the piece of furniture that target different regions of the body such as, for example, the lumbar region of a person's back, as well as lower back, mid-back, and upper back regions of the person's back.

### SUMMARY OF THE INVENTION

In accordance with the principles and teachings of the present invention, there has been developed a new and improved chair, sofa, or similar type of furniture piece wherein a lumbar actuator has been incorporated therein so as to engage the lumbar region of the back of a person sitting in the chair, sofa, or similar type of furniture piece. More particularly, the chair, sofa, or similar type of furniture piece, is provided with a main actuator which will conventionally move or actuate various parts of the furniture piece, such as, for example, the back portion of the furniture, and the footrest portion of the furniture piece. In addition, the furniture piece is provided with a headrest actuator which will move the headrest through predetermined incremental movements such that the headrest is disposed at a particular predetermined position as desired by the user, and a lumbar actuator which will move a lumbar support member to a predetermined extended position with respect to the person's lumbar region of the person's back, and will retract the lumber support member back to its original position. A first switch housing is provided upon a side portion of the furniture piece and is electronically connected to a control box so as to initiate movement of the various actuators so as to, in turn, move the various components of the furniture piece between extended and retracted positions. In connection with the lumbar actuator and the lumber support member, several different modes of operation are envisioned. For example, a first button can be depressed such that the lumbar actuator may simply be extended so as to, in turn, move the lumber support member to a predetermined extended position so as to provide necessary support and comfort to the lumbar region of the back of the person sitting in the furniture piece.

Alternatively, a second lumbar button, disposed upon a second side switch housing which is also located upon a side portion of the furniture piece, may be depressed such that the lumbar actuator may be cyclically extended and retracted in accordance with a predetermined frequency pattern so as to provide the user, seated in the furniture piece, with a massage that will promote blood circulation, comfort, and physical and mental relaxation. Still further, in accordance with a third mode of operation, when the second lumbar button is depressed, the lumbar actuator can be extended and retracted in accordance with varying degrees of frequency patterns similar to, for example, heated seats within modern automotive vehicles, so as to, in turn, achieve different modes of massage as may be desired by the person sitting in the furniture piece. More particularly, as is well known with respect to heated seat systems installed within modern automotive vehicles, when one depresses a button once, the heating mechanism for heating the seats is activated to a first degree, and if the button is depressed a second time, the heating mechanism for heating the seats is activated to a second degree. Likewise, if the button is depressed a third time, the heating mechanism for heating the seats is activated to a third degree, while depressing the button a fourth time terminates the heating system such that the seats are no longer heated. In a similar manner, depression of the second lumbar massage button can actuate the lumbar actuator in accordance with varying degrees of frequency such that the lumbar massage system can provide varying degrees of massage to the person seated in the chair or other piece of furniture so as to, in turn, achieve varying degrees of massage, comfort, and mental and physical relaxation as may be desired by the user. Still yet further, air bladder mechanisms, capable of being inflated and deflated in accordance with varying degrees of intensity and frequency, are also incorporated within the furniture piece and are located at predetermined strategic locations within the back portion of the furniture piece so as to, for example, provide massage therapy to a person's lower back, mid-back, and upper back regions. Lastly, the furniture piece also has a plurality of heating pads which are also located within the seat and back portions of the furniture piece, and which are also able to be regulated so as to achieve different degrees of heat, so as to provide heat therapy to the person's hip and back portions, it being appreciated that the new and improved heat and massage system of the present invention, comprising the variably cyclic lumbar massage, variably cyclic air bladder massage, and varying degrees of heat, provides the person, sitting in the furniture piece, with unique massage capabilities.

### BRIEF DESCRIPTION OF THE DRAWINGS

Various other features and attendant advantages of the present invention will be more fully appreciated from the following detailed description when considered in connection with the accompanying drawings in which like reference characters designate like or corresponding parts throughout the several views, and wherein:

FIG. 1 is a front, right side, top perspective view of a conventional PRIOR ART recliner chair illustrating one type of furniture piece within which the system of the present invention can be incorporated;

FIG. 2 is a schematic view of an AC power cable adapted to be electrically connected at one end thereof to a suitable source of AC power;

FIG. 3 is a schematic view of an AC/DC adapter which is adapted to be connected at one end thereof to a second end of the AC power cable, as illustrated within FIG. 2, for converting AC current to DC current;

FIG. 4 is a schematic view of a DC Y power cable which is adapted to be electrically connected at one end thereof to a second end of the AC/DC adapter, as illustrated within FIG. 3, for receiving DC current from the AC/DC adapter;

FIG. 5 is a schematic view of a main actuator which is adapted to be connected at one end thereof to a second end of the DC Y power cable, as illustrated within FIG. 4, so as to receive DC current from the DC Y power cable;

FIG. 6 is a schematic view of a headrest actuator which is adapted to be electrically connected to the main actuator as illustrated within FIG. 5;

FIG. 7 is a schematic view of a lumbar actuator which is adapted to be electrically connected to the main actuator as illustrated within FIG. 5 for providing lumbar support and/or massage to the lumbar region of the back of a person sitting in the piece of furniture as disclosed within FIG. 1,

FIG. 8 is a schematic view of an air compressor which is adapted to provide compressed air to a plurality of inflatable/deflatable air bladders so as to provide additional massage therapy to strategically located regions of the back of the person sitting in the piece of furniture as disclosed within FIG. 1;

FIG. 9 is a schematic view of a plurality of inflatable/deflatable air bladders which are adapted to be fluidically connected to the air compressor as illustrated within FIG. 8 so as to receive compressed air from the air compressor when inflation of the inflatable/deflatable air bladders is desired so as to provide massage therapy of strategically located regions of the back of the person sitting in the piece of furniture as illustrated within FIG. 1;

FIG. 10 is a schematic view of the back portion of the piece of furniture illustrating the strategic locations within which the air bladders, as illustrated within FIG. 9, can be located upon the back portion of the furniture piece, as illustrated within FIG. 1, so as to provide the person, sitting in the furniture piece illustrated within FIG. 1, desired massage therapy as a result of the air bladders being cyclically inflated and deflated in accordance with different intensities and frequencies;

FIG. 11 is a schematic view of a control box which is adapted to be electrically connected to the main actuator as illustrated within FIG. 5, and the air compressor as illustrated within FIG. 8;

FIG. 12 is a schematic view of a plurality of heating pads which are adapted to be located within the seat, back, and lumbar portions of the furniture piece, so as to provide heat therapy to the person's back, lumbar region, and hip portions, and which are also adapted to be electrically connected to the control box as illustrated within FIG. 11;

FIG. 13 is a schematic view of a first side switch housing which is adapted to be fixedly mounted upon a first side portion of the furniture piece, which is also adapted to be electrically connected to the control box as illustrated within FIG. 11, and wherein the first side switch housing comprises several buttons which, among other functions, may be depressed so as to control the various lumbar, headrest, and main actuators, as well as a USB port or interface for enabling the charging of various electronic devices, such as, for example, a mobile phone;

FIG. 14 is a schematic view of a second side switch housing which is adapted to be fixedly mounted upon a second side portion of the furniture piece, which is also adapted to be electrically connected to the control box as illustrated within FIG. 11, and wherein the second side switch housing also comprises several buttons which may be depressed so as to control the cyclical lumbar massage function of the lumbar actuator, as well as the cyclical inflation and deflation of the air bladders, the heating pads, LED indication lights so as to indicate the degree, level, or frequency at which the cyclical lumbar actuator and the air bladders are being operated, and a USB-TYPE C port or interface for enabling the charging of various electronic devices, such as, for example, a mobile phone;

FIG. 15 is a top plan view of a pair of cup holders which can be incorporated within the armrest portions of the piece of furniture and which have electrical connectors fixed thereto so as to receive electrical power whereby the cup holders may be illuminated by means of a plurality of LED lights;

FIG. 16 is a top plan view showing one of the cup holders, as disclosed within FIG. 15, mounted within one of the armrest portions of the piece of furniture and illuminated by means of the LED lights as a result of being electrically connected to a source of electrical power;

FIG. 17 is a front elevational view of an LED string light which can be fixedly connected to, for example, the lower edge portion of the footrest portion of the piece of furniture and which has an electrical connector fixed thereto so as to receive electrical power whereby the plurality of LED lights of the LED string light may be illuminated so as to provide ambience to the piece of furniture within, for example, a darkened room;

FIG. 18 is a front elevational of a piece of furniture, similar to the piece of furniture disclosed within FIG. 1, wherein the LED string light, as shown in FIG. 17, has in fact been fixedly connected to the lower edge portion of the footrest portion of a piece of furniture similar to the piece of furniture as illustrated within FIG. 1; and

FIG. 19 is a a schematic block diagram illustrating how all of the various components of the system, as illustrated within FIGS. 2-18, are electrically connected to each other in accordance with the principles and teachings of the present invention so as to provide the chair, as illustrated within FIG. 1, with the desired chair, headrest, lumbar movements, as well as the cyclical lumbar massage, cyclical air bladder massage, and heating therapy operations.

### DETAILED DESCRIPTION OF THE PREFERRED EMBODIMENT

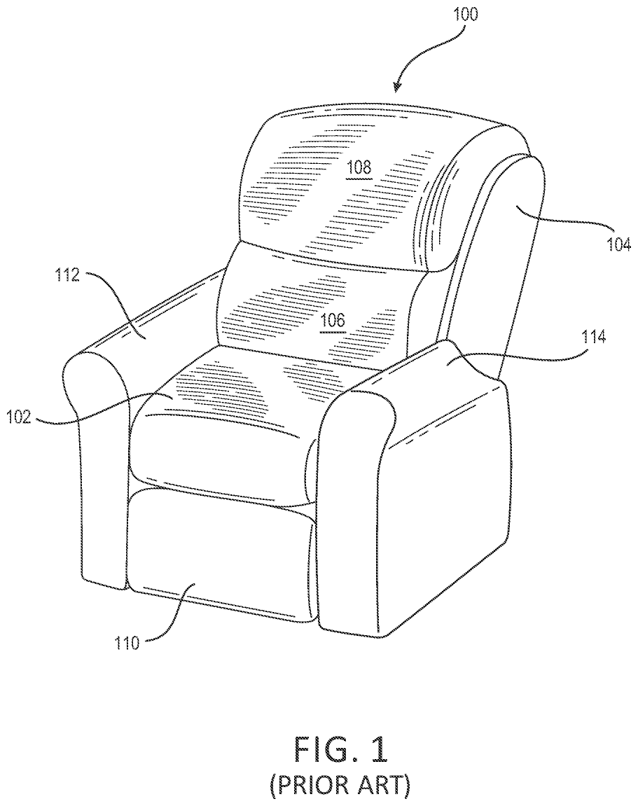

With reference first being made to FIG. 1, a conventional, PRIOR ART, lounger chair is disclosed and is generally indicated by the reference character 100. Chair 100 comprises a typical lounger chair which comprises a seat portion 102, a back portion 104 which may be moved between various positions between an upright position and a reclined position, a lumbar support portion 106 which is effectively part of the back portion 104 but which is positionally located so as to be capable of engaging the lumbar region of the back of the torso of the person seated in the chair 100, a headrest portion 108 which may be moved to various positions as desired by a person seated in the chair 100 such that the headrest portion 108 is disposed at a position that the person seated in the chair 100 finds most comfortable. In addition, the chair 100 further comprises a footrest portion 110 which, in a manner similar to that of the headrest portion 108, may be moved to various positions as desired by a person seated in the chair 100 such that the footrest portion 110 is disposed at a position that the person seated in the chair 100 finds most comfortable. Lastly, the chair 100 comprises a pair of oppositely disposed armrest portions 112,114.

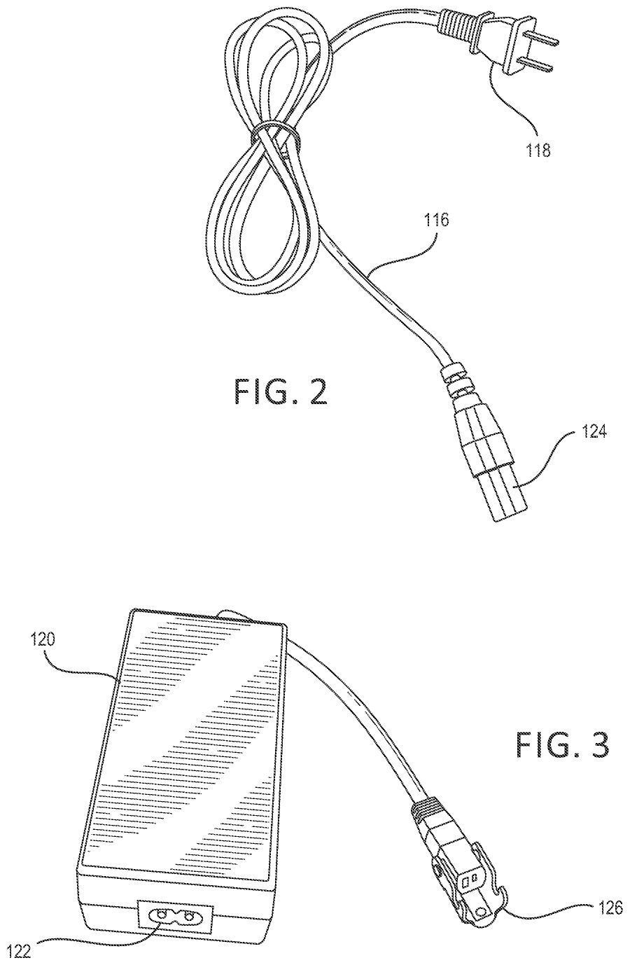

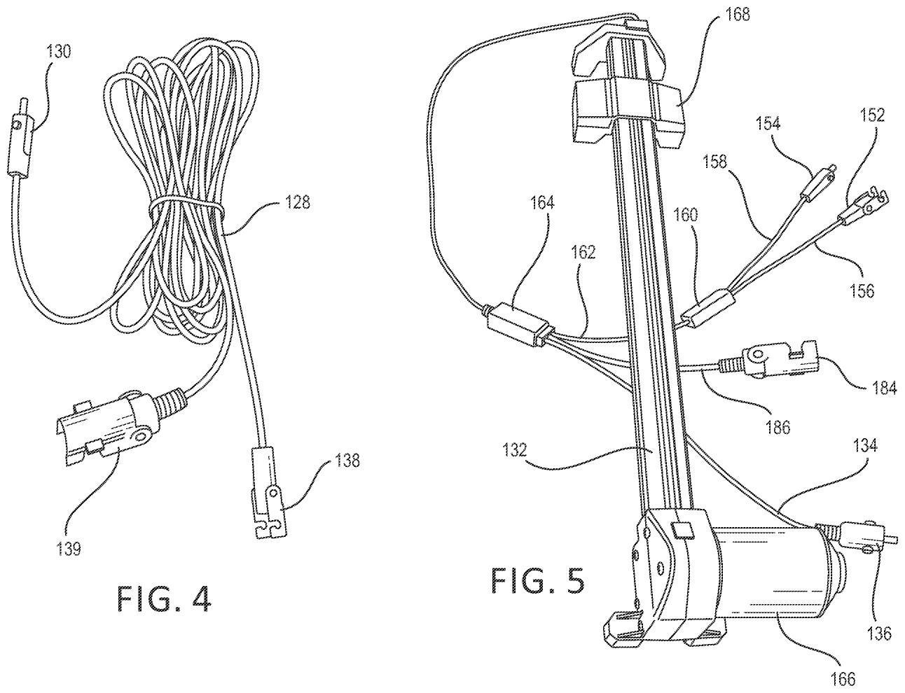

In accordance with the principles and teachings of the present invention, and with reference being made to FIGS. 2-10, there has been developed a new and improved chair, sofa, or other type of furniture piece, similar to the chair 100, wherein a heat and massage system has been incorporated therein so as to provide heat and massage therapy to various portions or regions of a person's back and hip regions as the person is sitting in the particular furniture piece. More particularly, the new and improved heat and massage system comprises the use of a lumbar actuator, a plurality of inflatable air bladders, and a plurality of heating pads strategically located within the back and lumbar portions 104,106 of the furniture piece, as well as the seat portion 102 of the furniture piece so as to provide heat therapy to the back, lumbar, and hip regions of the person sitting in the piece of furniture. More particularly, as can best be appreciated from FIGS. 2 and 3, the new and improved heat and massage system of the present invention comprises an AC power cable 116 which is adapted to be electrically connected at a first end thereof to a suitable source of AC power by means of a conventional plug 118, and an AC/DC adapter 120 which is provided with a socket 122 at a first end thereof to which a second end of the AC power cable 116 is adapted to be electrically connected by means of a suitable plug 124 which is to be inserted into the socket 122 of the AC/DC adapter 120, the AC/DC adapter 120 of course being provided to convert AC power or current to DC power or current. Accordingly, the AC/DC adapter 120 is provided with a suitable electrical connector 126 at a second opposite end thereof for outputting DC power from the AC/DC adapter. In turn, as can best be appreciated from FIG. 4, a DC Y power cable 128 is adapted to be electrically connected to the AC/DC adapter 120 by means of a suitable electrical connector 130, which is provided upon a first end of the DC Y power cable 128, wherein the electrical connector 130 of the DC Y power cable 128 is adapted to be electrically connected to the electrical connector 126 of the AC/DC adapter 120 so as to receive DC power or current from the AC/DC adapter 120.

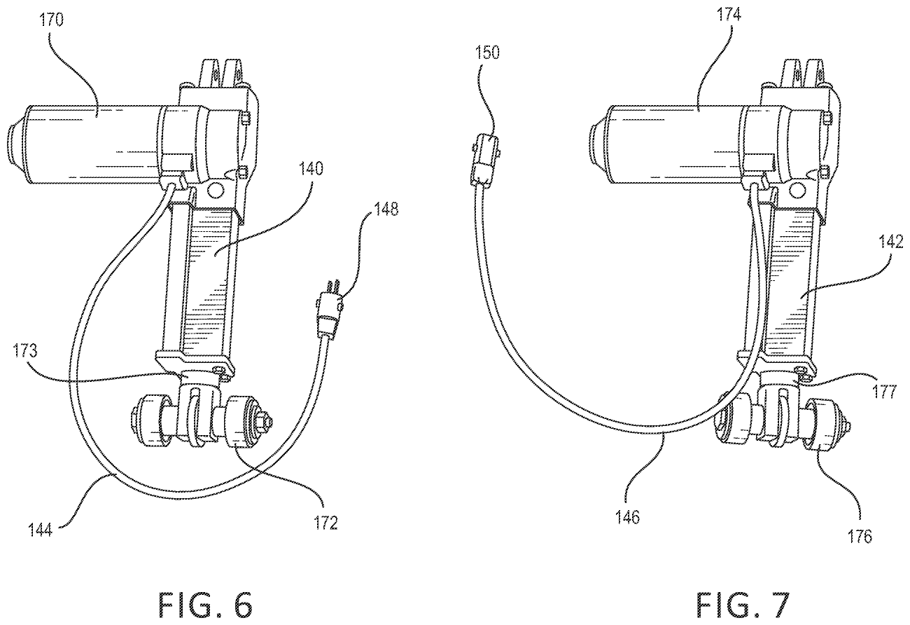

Continuing further, and with reference being made to FIG. 5, a main actuator 132 is disclosed, the main actuator 132 being provided for moving various components of the chair 100, such as, for example, the back portion 104 of the chair 100, as well as the footrest portion 110 of the chair 100. The main actuator 132 is provided with a power cable 134, at a first end of which there is provided a suitable electrical connector 136 for electrically connecting the main actuator 132 to the DC Y power cable 128 as a result of the electrical connector 136 of the main actuator power cable 134 being electrically connected to a suitable electrical connector 138 which is provided upon a second opposite end of the DC Y power cable 128, whereby the main actuator 132 can now receive DC current from the DC Y power cable 128. In turn, as can best be seen in FIGS. 6 and 7, a headrest actuator 140 and a lumbar actuator 142 are respectively disclosed. The headrest actuator 140 and the lumbar actuator 142 are respectively provided with power cables 144,146, and it is seen that the distal ends of the power cables 144,146 are respectively provided with suitable electrical connectors 148,150 which are adapted to be respectively connected to suitable electrical connectors 152,154 which are mounted upon distal end portions of a headrest actuator power cable 156 and a lumbar actuator power cable 158, as shown in FIG. 5.

In turn, the headrest actuator power cable 156 and the lumbar actuator power cable 158 are adapted to be electrically connected together by means of a first suitable junction connector 160 which is mounted upon a junction power cable 162 which, in turn, is adapted to be electrically connected to the main actuator power cable 134 by means of a second suitable junction connector 164. As can also be appreciated from FIGS. 4-6, the main actuator 132 comprises a drive motor 166, and a slider 168 which is adapted to be driven along the body of the main actuator 132 by means of the drive motor 166 so as to actuate or move the back portion 104 of the chair 100 and the footrest portion 110 of the chair 100, through means of conventional linkage systems, not shown, operatively connected to the slider 168. In a somewhat similar manner, it is seen that the headrest actuator 140 comprises a drive motor 170 and a pair of rollers or wheels 172 which are mounted upon the end of a telescopically movable tubular member 173, which are adapted to be engageable with the headrest 108, and which are adapted to be extended outwardly from an outer tube of the actuator 140 and retracted back into the outer tube of the actuator 140 by means of the drive motor 170, while the lumbar actuator 142 similarly comprises a drive motor 174 and a pair of rollers or wheels 176 which are mounted upon the end of a telescopically movable tubular member 177, which are adapted to be engageable with the lumbar portion 106, and which are adapted to be extended outwardly from an outer tube of the actuator 142 and retracted back into the outer tube of the actuator 142 by means of the drive motor 174.

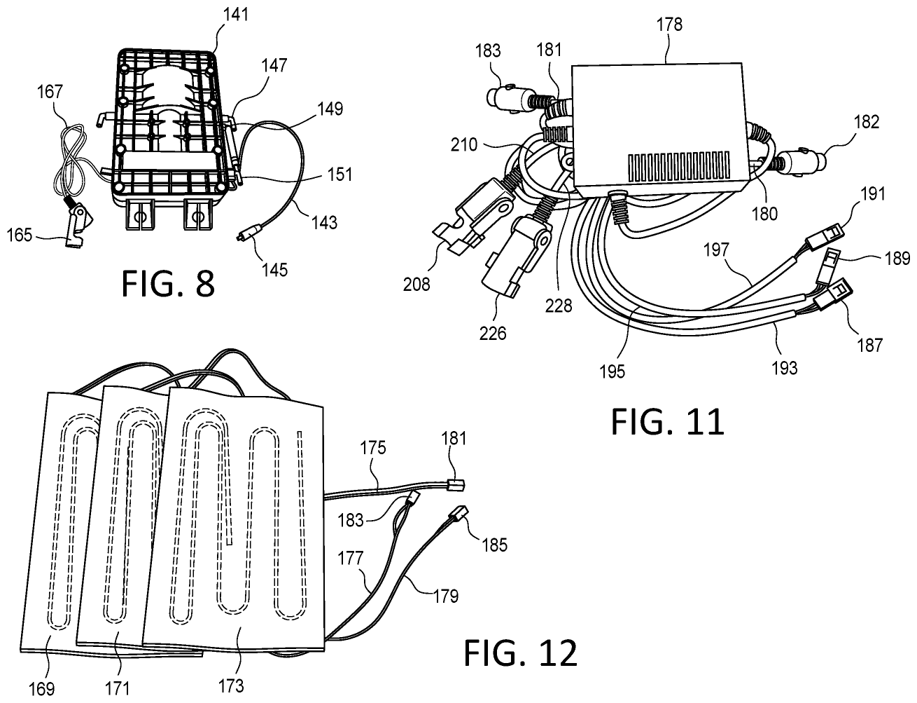

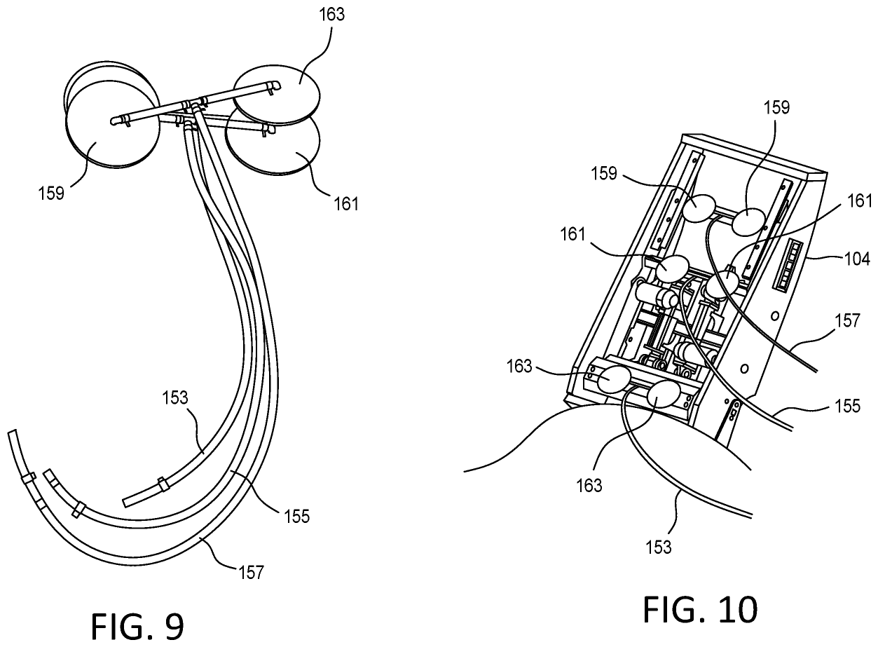

With reference again being made to FIG. 4, it is also seen that the DC Y power cable 128 further includes an electrical connector 139 which is adapted to be electrically connected to an air compressor 141 which is illustrated within FIG. 8 and which is provided with an air compressor power cable 143 having a suitable electrical connector 145 provided upon the distal end thereof for electrical connection to the electrical connector 139 of the DC Y power cable 128. The air compressor 141, in turn, is provided with a plurality of compressed air output ports 147,149,151 to which a plurality of air hoses 153,155,157, as illustrated within FIG. 9, are adapted to be connected such that compressed air can be respectively provided from the air compressor 141 to a plurality of inflatable/deflatable air bladders 159,161,163. As schematically illustrated within FIG. 10, the air bladders 159,161,163 may be strategically located at different positions throughout the back and lumbar portions 104,106 of the furniture piece 100 so as to provide the person, sitting in the furniture piece 100, with predetermined massage therapy directed toward specific portions or regions of the person's back. More particularly, as can be seen from FIG. 10, the first air bladder 159 actually comprises a pair of air bladders 159,159 which are located upon the upper back portion 104 of the furniture piece 100 and upon opposite sides thereof, that is, upon the left and right sides thereof, so as to impact, for example, the left and right shoulder blade portions or regions of the person's back, the second air bladder 161 actually comprises a pair of air bladders 161,161 which are located upon the middle back portion 104 of the furniture piece 100, or within the lumbar region 106 of the furniture piece, and upon opposite sides thereof, that is, upon the left and right sides thereof, so as to impact, for example, the left and right lumbar portions or regions of the person's back, while the third air bladder 163 actually comprises a pair of air bladders 163,163 which are located upon the lower back portion 104 of the furniture piece 100 and upon opposite sides thereof, that is, upon the left and right sides thereof and within or near the hip regions of the person sitting in the furniture piece 100, so as to impact, for examples, the left and right lower back or his regions of the person's back or hip regions. It is to be appreciated that the plurality of air hoses 153,155,157 will have different lengths so as to respectively be capable of pneumatic connection between the air compressor 141 and the air bladders 159,161,163 regardless of where the air bladders 159,161,163 are located upon the furniture piece.

With reference now being made to FIG. 11, there is disclosed a control box 178 which is adapted to be electrically connected to the main actuator 132, as illustrated within FIG. 5, by means of a first power cable 180 and a suitable electrical connector 182 which is adapted to be electrically connected to a suitable electrical connector 184 which is mounted upon the distal end of a power cable 186 which is electrically connected to the second junction connector 164 which leads to the main actuator 132, all as best seen in FIG. 5. In a similar manner, the control box 178 is also provided with a second power cable 181 and a suitable electrical connector 183 which is adapted to be electrically connected to a suitable electrical connector 165 disposed upon the distal end of a power cable 167 such that the air compressor 141 can be electrically connected to the control box 178. Continuing still further, and with reference being made to FIG. 12, a plurality of heating pads 169,171,173 are provided for heat therapy in accordance with the principles and teachings of the present invention, and it is seen that each one of the heating pads 169,171,173 is respectively provided with a power cable 175,177,179 upon the distal ends of which there are provided electrical connectors 181,183,185 for electrical connection to suitable electrical connectors 187,189,191 which are respectively fixedly mounted upon the distal ends of power cables 193,195,197 which are connected the control box 178 as seen in FIG. 11. The plurality of heating pads 169,171,173 are adapted to be located at predetermined locations of the back, lumbar, and seat portions 104,106,102 of the furniture piece so as to provide the person, sitting in the furniture piece 100, with predetermined heat therapy directed toward specific portions or regions of the person's back and hips.

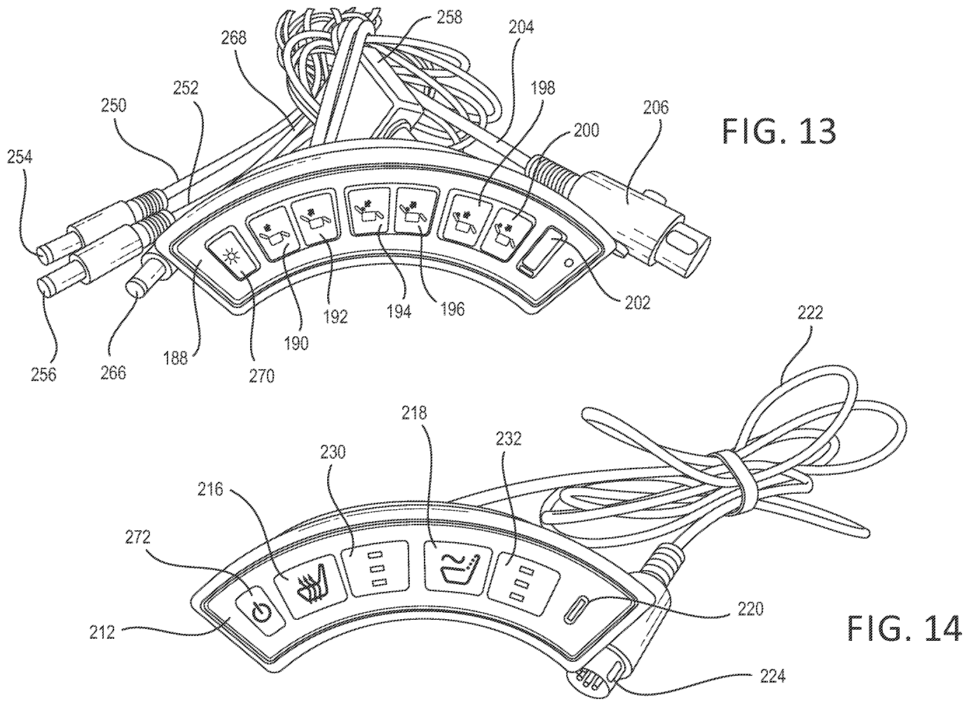

With reference now being made to FIG. 13, a first side switch housing 188 is disclosed and is seen to comprise a plurality of various different buttons 190,192,194,196,198,200, the functions of which will be discussed shortly hereinafter, as well as a USB port 202 which is provided so as to provide a charging port for mobile devices such as, for example, a mobile phone. The first side switch housing 188 is adapted to be mounted upon a first side portion of the furniture piece or chair 100 as illustrated within FIG. 1, and is provided with an electrical output signal cable 204 which has a suitable electrical connector 206 mounted upon the distal end thereof for electrical connection to a complimentary electrical connector 208 which is mounted upon the distal end of a power cable 210 which is connected to the control box 178 as seen in FIG. 11. In this manner, electrical signals can be sent to the control box 178 when the various buttons 190,192,194,196.198,200 are depressed, as will be discussed more fully hereinafter, whereby, in turn, the control box 178 can then route control signals to the main actuator 132, the headrest actuator 140, and the lumbar actuator 142.

With reference now being made to FIG. 14, a second side switch housing 212 is disclosed and is likewise seen to comprise a plurality of various different buttons 216,218, the functions of which will be discussed shortly hereinafter, as well as a USB-TYPCE C port 220 which is provided so as to provide a charging port for mobile devices such as, for example, a mobile phone. The second side switch housing 212 is adapted to be mounted upon a second side portion of the furniture piece or chair 100 as illustrated within FIG. 1, and is provided with an electrical output signal cable 222 which has a suitable electrical connector 224 mounted upon the distal end thereof for electrical connection to a complimentary electrical connector 226 which is mounted upon the distal end of a power cable 228 which is connected to the control box 178 as seen in FIG. 11. In this manner, electrical signals can be sent to the control box 178 when the various buttons 216,218 are depressed, as will be discussed more fully hereinafter, whereby, in turn, the control box 178 can then route control signals to the air bladders 159,161,163, as well as to the lumbar actuator 142, whereby the air bladders 159,161,163 and the lumbar actuator 142 will be operated in cyclical modes so as to provide different massage therapies to the person sitting in the furniture piece or chair 100. In addition, it is also seen that the second side switch housing 212 also includes a first set of indicator lights 230 which are operatively associated with the button 216, and a second set of indicator lights 232 which are operatively associated with the button 218, all of which will be explained more fully hereinafter.

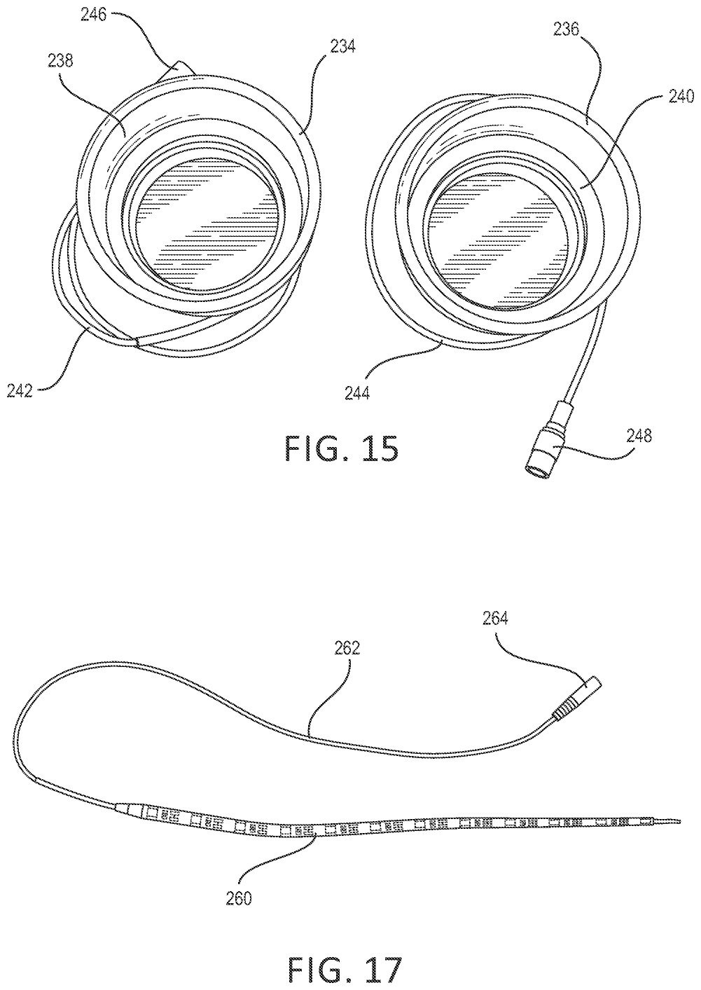

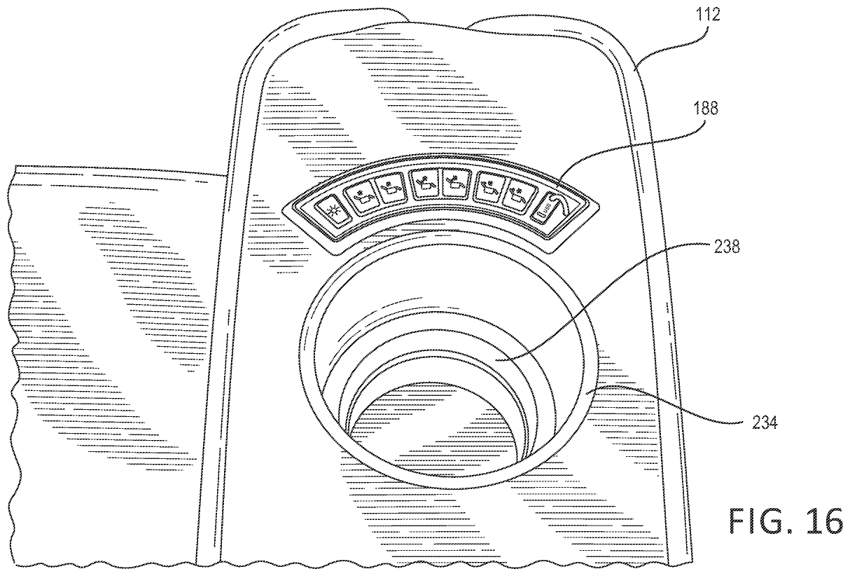

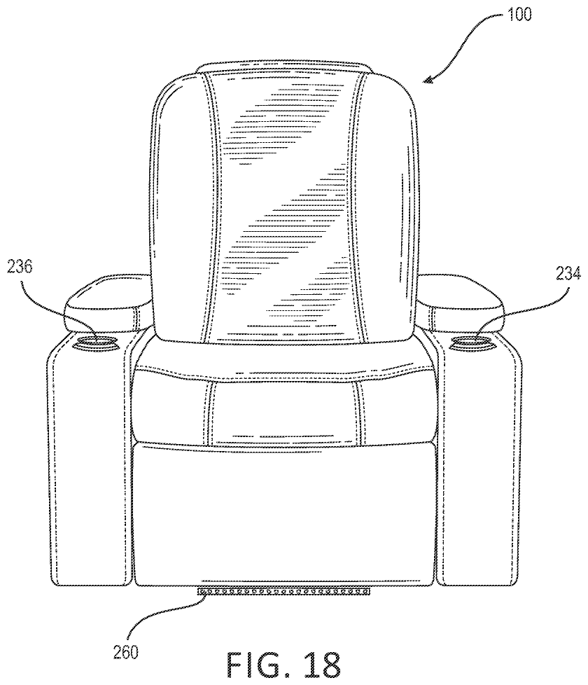

With reference now being made to FIGS. 15 and 16, a pair of cup holders 234,236 are disclosed within FIG. 15, and one of the cup holders 234 is disclosed in FIG. 16 as being mounted, for example, within an armrest portion 112 of the piece of furniture or chair 100. A plurality of LED lights 238,240 are respectively provided within the bottom portion of each cup holder 234,236 so as to illuminate the same when the cup holders 234,236 are respectively mounted within the armrest portions 112,114 of the piece of furniture or chair 100, and in order to supply electrical power to the LED lights 238,240, the cup holders 234, 236 are respectively provided with power cables 242,244 which have suitable electrical connectors 246,248 mounted upon the distal end portions thereof for electrical connection to the first side switch housing 188, as seen in FIG. 13, by means of power cables 250,252 which are provided with suitable electrical connectors 254,256. It is to be noted that the power cable 204, which leads to the control box 178, and the power cables 254,256, are all electrically connected to a suitable power junction box 258, as is also seen within FIG. 13. Continuing further, in a similar manner, and with reference being made to FIGS. 17 and 18, an LED light strip is disclosed at 260 and is electrically connected to a power cable 262 which has a suitable electrical connector 264 mounted upon the distal end thereof for electrical connection to a suitable electrical connector 266 which is mounted upon the distal end of a power cable 268 which is electrically connected to the power junction box 258, all as disclosed within FIG. 13. In FIG. 18, the LED light strip 260 is disclosed as being affixed to the bottom edge portion of the footrest portion 110 of the piece of furniture of chair 110. As is also disclosed within FIG. 13, a button 270 is provided upon the first side switch housing 188 for turning the LED light strip 260, as well as the LED lights 238,240 disposed within the bottom portions of the cup holders 234,236, ON and OFF. In a similar manner, it is seen that the second side switch housing 212 is provided with a power button or switch 272 so as to electronically activate the entire system of the present invention.

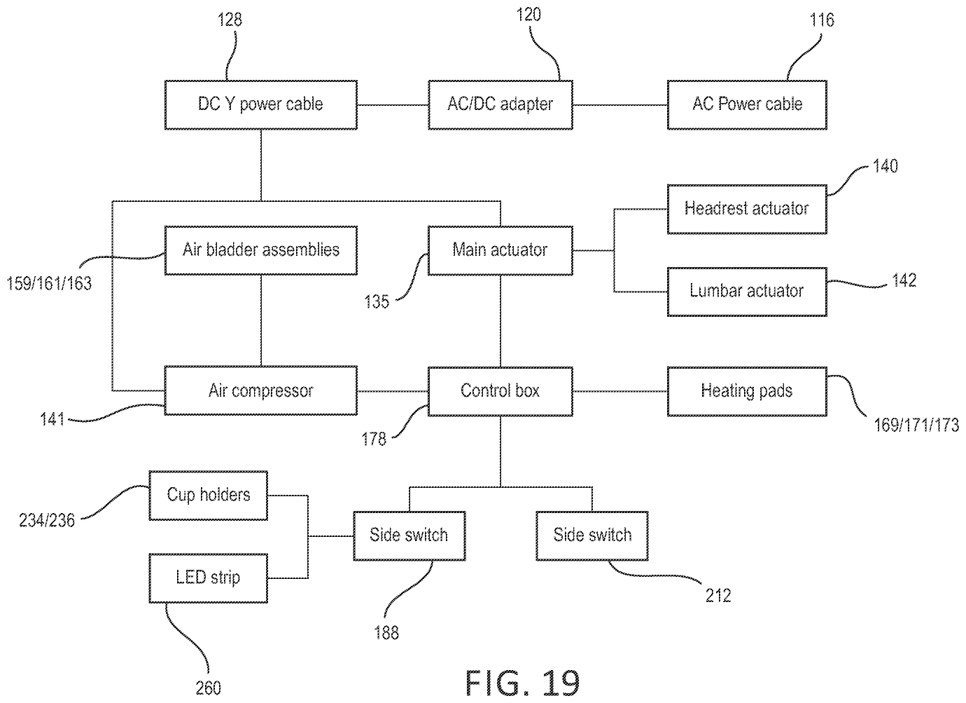

With reference lastly being made to FIG. 19, there is disclosed a block diagram which schematically illustrates how all of the various components of the system, as illustrated within FIGS. 2-18, are electrically connected to each other in accordance with the principles and teachings of the present invention so as to provide the chair 100, as illustrated within FIGS. 1 and 18, with the desired chair, headrest, and lumbar movements, as well as the lumbar massage, air bladder massage, and heat therapy operations.

Having described all of the operative components of the system of the present invention, a brief description of how the various components operate so as to achieve the objectives of the present invention will now be provided. More particularly, with reference first being made to FIGS. 13 and 14, the first and second side switch housings are disclosed at 188,212 respectively, however, it is to be appreciated that the control buttons disposed upon the first and second side switch housings 188,212 may be disposed upon either one of the first and second side switch housings 188,212, that is, the various control buttons disposed upon the first side switch housing 188 may be disposed upon the second side switch housing 212, and vice versa, or alternatively, it is to be appreciated further that all of the control buttons may be disposed upon a single side switch housing. In any case, and more particularly, it is seen, for example, the power button 272 disposed upon the second side switch housing 212 is to be depressed or activated first so as to electronically power-up the entire system. Subsequently, button 216, disposed upon the second side switch housing 212, may be depressed once so as to send a first signal to the control box 178 such that a control signal is transmitted to the air compressor 141 so as to control the actuation of the air bladders 159,161,163 whereby the air bladders 159,161,163 will be inflated and deflated to a first predetermined degree in accordance with a first predetermined repetitively cyclic mode of operation so as to effectively provide massage therapy with a first degree of frequency and intensity.

In a similar manner, the depression of button 216 will likewise transmit a signal to the main actuator 132 such that a signal may, in turn, be transmitted to the lumbar actuator 142 such that the lumbar actuator 142 will extend to a first length or degree and in accordance with a first predetermined repetitive cyclic mode of operation so as to effectively provide massage therapy within the lumbar region of the person's back in accordance with a first degree of frequency and intensity. Concomitantly with the depression of the button 216 once, upon the second switch housing 212, a first one of the set of indicator lights 230 will be illuminated a predetermined color, such as, for example, blue, so as to indicate to the person sitting in the piece of furniture or chair 100 that their bladders 159,161,163, as well as the lumbar actuator 142, have been activated in accordance with their first modes of operation. If the button is depressed a second or third time, the air bladders 159,161,163 will be inflated and deflated to second and third predetermined extents, within second and third predetermined times, so as to effectively achieve or provide massage therapy with second and third degrees of frequency and intensity. In a similar manner, the lumbar actuator 142 will be extended and retracted to second and third predetermined extents, within second and third predetermined times, so as to likewise effectively achieve or provide massage therapy with second and third degrees of frequency and intensity. It is also to be noted that second and third lights of the set of indicator lights 230 will be illuminated so as to again indicate to the person sitting in the piece of furniture or chair 100 that the bladders 159,161,163, as well as the lumbar actuator 142, have been activated in accordance with their second and third modes of operation. If the button 216 is depressed a fourth time, the air bladders 159,161,163 will be deactivated, as will the lumbar actuator 142, and the set of indicator lights 230 will be extinguished. In a similar manner, the depression of the button 218 controls the activation of the heating pads 169,171, 173, whereby, in a manner similar to the actuation of the button 216, when the button 218 is depressed once, a signal is transmitted to the control box 178 such that a signal is then transmitted from the control bx 178 to the heating pads 169,171,173 whereby the same are activated to first predetermined heating level, and a first one of the set of indicator lights 232 will be illuminated a predetermined color, such as, for example, red, so as to indicate to the person sitting in the furniture piece or chair 100, that the heating pads 169, 171,173 have in fact been activated to a first heating mode of operation. Depression of the button 218 second and third times indicate that the heating pads 169,171,173 have been activated to second and third heating levels, whereby second and third lights of the second set of indicator lights 232 will be illuminated so as to confirm to the person sitting in the furniture piece or chair that the heating pads 169,171,173 have in fact been activate to their second and third heating level modes of operation. Depression of the button 218 will terminate the activation of the heating pads 169,171,173 and will extinguish all lights comprising the second set of indicator lights 232. Likewise, depression of the power switch 272 deactivates the entire massage system.

With reference now being made to FIG. 13, when button 270 is depressed, power is supplied to the LED lights 238 operatively associated with the cup holders 234,236, as well as to the LED light strip 260. Depression of the button 270 a second time extinguishes the LED lights 238,260. Continuing further, when button 192 is depressed, a signal is transmitted to the control box 178 so as to actuate the main actuator 132 which, in turn, actuates the back portion 104 of the furniture piece or chair 100, as well as the footrest portion 110 of the furniture piece or chair 100. Conversely, when button 190 is depressed, the back portion 104 104 of the furniture piece or chair 100, as well as the footrest portion 110 of the furniture piece or chair 100, are returned to their original, undeployed positions. Button 196 controls the lumbar actuator 142 whereby when button 196 is depressed, button 196 transmits a signal to the control box 178 which, in turn, routes a signal to the lumbar actuator 142 through the main actuator 132, whereby the lumbar actuator 142 is extended so as to provide support to the lumbar portion 106 of the furniture piece or chair 100. Conversely, depression of the button 194 retracts the lumbar actuator 142. In a similar manner, when button 200 is depressed, button 200 transmits a signal to the control box 178 which, in turn, routes a signal to the headrest actuator 140 through the main actuator 132, whereby the headrest actuator 140 is extended so as to provide desired support and comfort to the head of the person sitting in the furniture piece or chair 100. Conversely, when button 198 is depressed, the headrest is returned to its normal, undeployed position. It is lastly to be noted that since control buttons 196,194 control the extension and retraction of the lumbar actuator 142, if either of the control buttons 196,194 is depressed while the lumbar actuator 142 is moving the lumbar support portion 106 of the furniture piece or chair 100 in accordance with any one of the various massage operating modes, as has been fully discussed hereinbefore, the movement of the lumbar actuator 142 will be temporarily disabled or terminated and will be reactivated or enabled when either one of the control buttons 196,194 is released.

Obviously, many variations and modifications of the present invention are possible in light of the above teachings. It is therefore to be understood that within the scope of the appended claims, the present invention may be practiced otherwise than as specifically described herein.

What is claimed is:

1. A heat and massage system for a piece of furniture, comprising:
a seat portion;
a back portion;
a lumbar support portion;
a lumbar actuator operatively connected to said lumbar support portion of said piece of furniture for extending and retracting said lumbar support portion of said piece of furniture to a predetermined extent and in accordance with a predetermined repetitively cyclic mode of operation so as to effectively provide massage therapy to the lumbar region of a person's back, with a predetermined amount of intensity and frequency, when the person is seated upon said piece of furniture.

2. The massage system as set forth in claim 1, wherein:
said lumbar actuator is operatively connected to said lumbar support portion of said piece of furniture so as to extend and retract said lumbar support portion of said piece of furniture in accordance with a plurality of different repetitively cyclic modes of operation so as to effectively provide different massage therapy modes of operation of varying frequency to the lumbar region of a person's back when the person is seated upon said piece of furniture.

3. The massage system as set forth in claim 2, further comprising:
a control box mounted within said piece of furniture; and
a first side switch housing mounted upon a side of said furniture piece and comprising a first button which, when depressed a predetermined number of times, transmits signal to said control box which, in turn, transmits signals to said lumbar actuator so as to actuate said lumbar support portion of said piece of furniture in accordance with predeterminedly different repetitive cyclic modes of operation so as to effectively provide massage therapy of varying frequency to the lumbar region of a person's back when the person is seated upon said piece of furniture.

4. The massage system as set forth in claim 3, wherein:
said lumbar actuator is operatively connected to said lumbar support portion of said piece of furniture so as to extend and retract said lumbar support portion of said piece of furniture in accordance with a plurality of predeterminedly different repetitively cyclic modes of operation comprising different lengths of movement of said lumbar actuator so as to effectively provide different massage therapy modes of operation of varying intensity to the lumbar region of a person's back when the person is seated upon said piece of furniture.

5. The massage system as set forth in claim 3, further comprising:
a control box mounted within said piece of furniture; and
a side switch housing mounted upon a side of said furniture piece and comprising a first button which, when depressed a predetermined number of times, transmits signal to said control box which, in turn, transmits signals to said lumbar actuator so as to actuate said lumbar support portion of said piece of furniture in accordance with predetermined repetitive cyclic modes of operation so as to effectively provide massage therapy of varying frequency and intensity to the lumbar region of a person's back when the person is seated upon said piece of furniture.

6. The massage system as set forth in claim 5, further comprising:
a first set of indicator lights mounted upon said side switch housing for indicating said predetermindedly different repetitive cyclic modes of operation.

7. The massage system of claim 1, further comprising:
at least one heating implement operatively connected to said back portion of said piece of furniture and said seat portion of said furniture piece for providing heat therapy to a person's back, lumbar, and hip regions of the person when the person is seated upon said piece of furniture in addition to said massage therapy impressed upon the person's back by means of said lumbar actuator operatively connected to said lumbar support portion of said piece of furniture.

8. The massage system as set forth in claim 7, wherein:
said at least one heating implement comprises a plurality of heating implements connected to said back, lumbar support, and seat portions of said piece of furniture so as to provide heat therapy to back, lumbar, and hip regions of the person when the person is seated upon said piece of furniture in addition to the massage therapy provided to the person seated in the piece of furniture by said lumbar actuator operatively connected to said lumbar support portion of said piece of furniture.

9. The massage system as set forth in claim 8, further comprising:
a control box mounted within said piece of furniture; and
a side switch housing mounted upon a side of said furniture piece and comprising a second button which, when depressed a predetermined number of times, transmits signal to said control box which, in turn, transmits signals to said plurality of heating implements so as to activate said plurality of heating implements to predeterminedly different levels of heat.

10. The massage system as set forth in claim 9, further comprising:
a second set of indicator lights mounted upon said side switch housing for indicating said predetermindedly different levels of heat.

11. The massage system as set forth in claim 8, wherein:
said plurality of heating implements comprise a plurality of heating pads.

12. An auto cycle lumbar massage system for a piece of furniture, comprising:
a seat portion;
a back portion;
a lumbar support portion;
a lumbar actuator operatively connected to said lumbar support portion of said piece of furniture for extending and retracting said lumbar support portion of said piece of furniture in accordance with a predetermined repetitive cyclic mode of operation so as to effectively provide massage therapy to the lumbar region of a person's back when the person is seated upon said piece of furniture; and
at least one inflatable/deflatable air bladder implement operatively connected to said back portion of said piece of furniture for inflation and deflation of said at least one inflatable/deflatable air bladder implement in accordance with a predetermined repetitive cyclic mode of operation so as to effectively provide massage therapy to at least one region of a person's back when the person is seated upon said piece of furniture in addition to said massage therapy provided to the person's back by said lumbar actuator.

13. The system as set forth in claim 12, wherein:
said at least one inflatable/deflatable air bladder implement comprises a plurality of inflatable/deflatable air bladder implements strategically located upon said back and lumbar support portions of said piece of furniture so as to provide massage therapy to strategically located regions of the person's back when the person is seated upon said piece of furniture in addition to the massage therapy provided to the person seated in the piece of furniture by said lumbar actuator operatively connected to said lumbar support portion of said piece of furniture.

14. The system as set forth in claim 13, comprising:
an air compressor for supplying compressed air to said plurality of inflatable/deflatable air bladder implements.

15. The system as set forth in claim 1, further comprising:
a footrest portion; and
a main actuator operatively connected to said back portion and said footrest portion for moving said footrest and back portions of said furniture piece toward extended and retracted positions.

16. The system as set forth in claim 1, further comprising:
a headrest portion; and
a headrest actuator operatively connected to headrest portion for moving said headrest of said furniture piece toward extended and retracted positions.

17. The system as set forth in claim 1, further comprising:
a USB port provided upon said side switch housing for permitting mobile devices to be connected to said USB port in order to recharge said mobile devices.

18. The system as set forth in claim 1, further comprising:
a USB TYPE-C port provided upon said side switch housing for permitting mobile devices to be connected to said USB TYPE-C port in order to recharge said mobile devices.

19. The system as set forth in claim 1 wherein:
said piece of furniture comprises a lounger chair.

20. The system as set forth in claim 1, further comprising:
at least one cup holder defined within an armrest portion of said piece of furniture;
at least one set of LED lights operatively associated with said at least one cup holder of said piece of furniture; and
at least one LED string light operatively associated with said piece of furniture so as to provide an illuminated outline to said piece of furniture.
